(19) United States
(12) Patent Application Publication
(10) Pub. No.: US 2026/0215707 A1
(43) Pub. Date: Jul. 30, 2026

(54) PROBE FOR PULSE OXYMETERS

(71) Applicant: STANLEY ELECTRIC CO., LTD., Meguro-ku, Tokyo (JP)

(72) Inventors: Hiroto FUKUSHIMA, Tokyo (JP); Tsukasa IMURA, Tokyo (JP)

(73) Assignee: STANLEY ELECTRIC CO., LTD., Meguro-ku, Tokyo (JP)

(21) Appl. No.: 19/141,950

(22) PCT Filed: Dec. 18, 2023
(86) PCT No.: PCT/JP2023/045369

§ 371 Date: Jun. 20, 2025

(30) Foreign Application Priority Data
Dec. 22, 2022 (JP) 2022-205090

(51) Int. Cl. A61B 5/1455; A61B 5/00

(52) U.S. Cl. CPC A61B 5/14552; A61B 5/6826; A61B 2560/0462; A61B 2562/0238; A61B 2562/185

(57) ABSTRACT

A probe includes a ring-shaped base material to be attached to a finger of a subject, a flexible film mounted on an inner circumferential surface of the ring-shaped base material, and a light-emitting element and a light-receiving element mounted on the film at positions facing each other across a finger of the ring-shaped base material. The ring-shaped base material has a C-shaped cross section and is flexible. The light-emitting element and the light-receiving element are both bare chips, and are directly bonded to wiring provided on a surface of the film. The surroundings of light-emitting element and the light-receiving element are sealed with a transparent sealing material.

### TECHNICAL FIELD

The present invention relates to a probe for a pulse oximeter, the probe including a light source section and a light-receiving section.

### BACKGROUND ART

In a pulse oximeter, a probe is attached to a fingertip or the like, light (red light and infrared light) is emitted onto the fingertip or the like from a light-emitting section within the probe, and the light that passes through the fingertip or the like is received by a light-receiving section in the probe. Accordingly, the presence ratio between oxyhemoglobin, which is bound to oxygen, and reduced hemoglobin, which is not bound to oxygen, among the hemoglobin in the blood is measured by utilizing the difference in absorption rates of red light and infrared light.

In medical settings, disposable-type probes are often used to prevent infection. In a disposable-type probe, a light-emitting section and a light-receiving section are disposed on the inner side of an adhesive tape of a size that covers a finger, and the adhesive surface of the adhesive tape is affixed to the fingertip to be fixed and used. In commercially available probes for a pulse oximeter, a rigid circuit board such as glass epoxy, on which a pre-packaged LED is mounted, is used as the light-emitting section. A photodiode is used for the light-receiving section. These are respectively mounted on the adhesive tape.

PTL 1 describes a problem in which heat generated by the LED becomes trapped on the inner side of the adhesive tape, causing the body temperature of the subject at the area where the probe is attached to rise by 5 to 6°C., and posing a risk that the subject suffers low-temperature burns. In order to solve this problem, PTL 1 proposes a probe that uses a flexible holding material (adhesive tape) with good thermal conductivity.

### CITATION LIST

### Patent Literature

PTL 1: JP3156114B

### SUMMARY OF INVENTION

### Technical Problem

The probe for a pulse oximeter in PTL 1 attempts to suppress the accumulation of heat from the light-emitting section by using a holding material (adhesive tape) with good thermal conductivity, and to solve the problem of the light-emitting section protruding from the adhesive tape by the flexibility of the holding material. However, the size of the light-emitting section, in which a pre-packaged LED is mounted on a rigid circuit board such as glass epoxy, protrudes by 1.5 to 3 mm in height from the adhesive surface of the adhesive tape. Therefore, it is difficult to completely absorb the protrusion of the light-emitting section by the flexibility of the adhesive tape.

When the protruding light-emitting section and light-receiving section are pressed against the finger by the adhesive tape, blood flow becomes stagnant, making it difficult for the heat from the light-emitting section to be dissipated to the surroundings by the blood flow, resulting in an increase in the temperature of the finger. Due to the interaction between compression of the finger and temperature increase, marks of the light-emitting section or the light-receiving section are likely to form on the skin of the finger, or skin irritation is likely to occur, and there is a risk that low-temperature burns may occur when attached for a long time. Therefore, medical professionals must frequently replace the probe to avoid low-temperature burns—approximately every eight hours for adults, and less than eight hours for newborns, elderly patients, and patients with sensitive skin—which places a burden on medical professionals.

Furthermore, when attaching a conventional probe for a pulse oximeter, it is necessary to accurately align the optical axes of the light-emitting section and the light-receiving section across the finger, and to affix the adhesive tape to the finger. Therefore, the task of aligning the probe when replacing the probe also places a burden on medical staff.

An object of the present invention is to provide a probe for a pulse oximeter in which the light-emitting section and the light-receiving section do not protrude, temperature rise in the light-emitting section can be suppressed, and alignment of the light-emitting section and the light-receiving section is not required when attaching the probe to a subject.

### Solution to Problem

In order to achieve the above-mentioned object, the present invention provides a probe for a pulse oximeter having a ring-shaped base material to be attached to a finger of a subject, a flexible film mounted on the inner circumferential surface of the ring-shaped base material, and a semiconductor light-emitting element and a semiconductor light-receiving element mounted on the film at positions facing each other across the finger of the ring-shaped base material. The ring-shaped base material is partially cut out in the circumferential direction, has a C-shaped cross section, and is flexible. Both the semiconductor light-emitting element and the semiconductor light-receiving element are bare chips including a semiconductor layer and a pair of electrodes provided on the semiconductor layer. A pair of electrodes of the semiconductor light-emitting element and the semiconductor light-receiving element are directly bonded to wiring provided on the surface of the film. The surroundings of the semiconductor light-emitting element and the semiconductor light-receiving element are each sealed with a sealing material that transmits light emitted by the semiconductor light-emitting element. The semiconductor light-emitting element emits light toward the finger, and the semiconductor light-receiving element receives the light that is emitted from the semiconductor light-emitting element and passes through the finger.

### Advantageous Effects of Invention

The probe for a pulse oximeter of the present invention has the light-emitting section and the light-receiving section not protruding from the film, and since the light-emitting section is a bare chip, the thermal conductivity is good and the temperature rise can be suppressed, and optical axis alignment between the light-emitting section and the light-receiving section is unnecessary when attaching the probe to the subject.

### BRIEF DESCRIPTION OF DRAWINGS

FIG. 1(a) is a top view of a probe for a pulse oximeter according to Embodiment 1, and FIG. 1(b) is a front view.

FIGS. 2(a) and 2(b) are cross-sectional views of the probe for a pulse oximeter according to Embodiment 1 of the present invention.

FIG. 3 is a view showing one manufacturing process of the probe for a pulse oximeter of Embodiment 1.

FIGS. 4(a) to 4(c) are views for describing that the optical axis shifts depending on the size of a finger when the probe for a pulse oximeter of Embodiment 1 is attached to the finger, and are views showing one manufacturing process.

FIGS. 5(a) and 5(b) are views for describing that the optical axis shifts depending on the size of the finger when the probe for a pulse oximeter of Embodiment 1 is attached to the finger.

FIGS. 6(a) and 6(b) are views for describing that the optical axis shifts depending on the size of the finger when the probe for a pulse oximeter of Embodiment 1 is attached to the finger.

FIGS. 7(a) to 7(d) are views showing the manufacturing process of the probe for a pulse oximeter of Embodiment 1.

FIGS. 8(a) to 8(f) are explanatory views showing the manufacturing process of the probe for a pulse oximeter of Embodiment 1.

FIGS. 9(a) to 9(e) are explanatory views showing the manufacturing process of the probe for a pulse oximeter of Embodiment 1.

FIGS. 10(a-1) to 10(d-1), 10(a-2) to 10(d-2), and 10(a-3) to 10(c-3) are explanatory views showing the manufacturing process of the probe for a pulse oximeter of Embodiment 1.

FIGS. 11(a) and 11(b) are cross-sectional views of a probe for a pulse oximeter according to Embodiment 2 of the present invention.

FIG. 12 is a view showing one manufacturing process of the probe for a pulse oximeter of Embodiment 2.

FIG. 13 is a view showing one manufacturing process of the probe for a pulse oximeter of Embodiment 2.

FIGS. 14(a) and 14(b) are cross-sectional views showing a peripheral structure and an optical path of a semiconductor light-receiving element 31 of probes of Modification Examples 1 and 2.

FIGS. 15(a) and 15(b) are cross-sectional views showing a peripheral structure and an optical path of the semiconductor light-receiving element 31 of probes of Modification Examples 3 and 4.

FIGS. 16(a) and 16(b) are cross-sectional views showing a peripheral structure and an optical path of the semiconductor light-receiving element 31 of probes of Modification Examples 5 and 6.

FIGS. 17(a) and 17(b) are cross-sectional views of a probe for a pulse oximeter according to Embodiment 3 of the present invention.

FIG. 18 is a cross-sectional view showing a peripheral structure and an optical path of the semiconductor light-receiving element 31 of the probe of Embodiment 3 of the present invention.

FIG. 19 is a cross-sectional view showing a peripheral structure and an optical path of the semiconductor light-receiving element 31 of a probe of Modification Example 8.

FIG. 20 is a cross-sectional view showing a peripheral structure and an optical path of the semiconductor light-receiving element 31 of a probe of Modification Example 9.

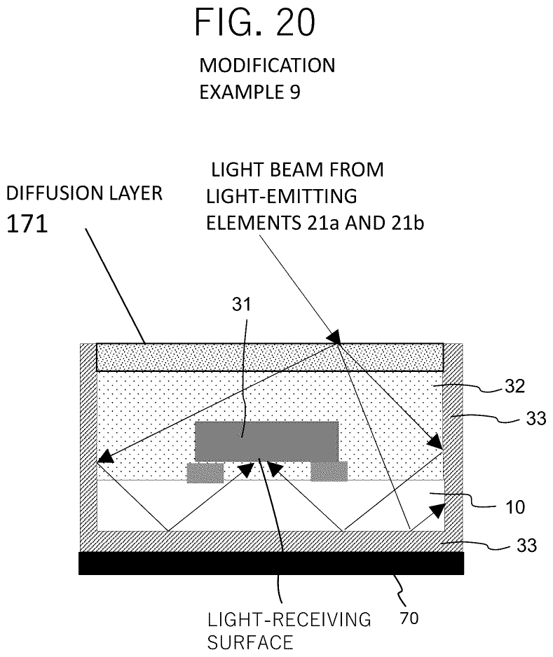

FIGS. 21(a) and 21(b) are cross-sectional views showing a peripheral structure and an optical path of the semiconductor light-receiving element 31 of probes of Modification Examples 10 and 11.

FIGS. 22(a) to 22(e) are views for describing a manufacturing process for the peripheral structure of the semiconductor light-receiving element 31 of a probe of Modification Example 11.

FIG. 23 is a cross-sectional view of a probe according to Modification Example 12.

### DESCRIPTION OF EMBODIMENTS

One embodiment of the present invention will be described below with reference to the drawings.

### Embodiment 1

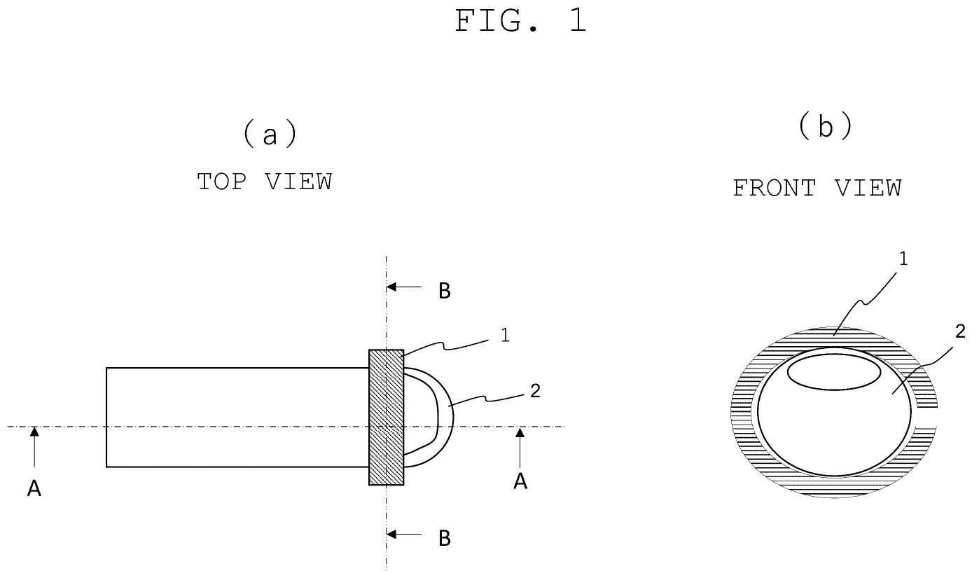

The structure of a probe for a pulse oximeter 1 according to Embodiment 1 will be described with reference to FIGS. 1 to 3.

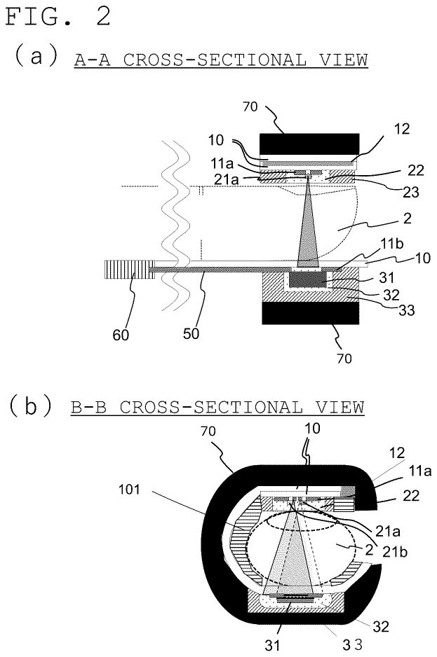

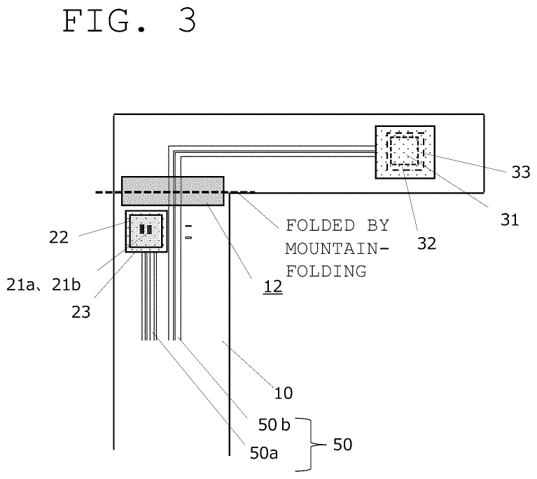

FIGS. 1(a) and 1(b) are a top view and a front view of the probe for a pulse oximeter 1 according to Embodiment 1. However, in FIG. 1, although the probe 1 is not a cross-sectional view, hatching is provided for clarity. FIGS. 2(a) and 2(b) are sectional views taken along lines A-A and B-B of the probe 1. FIG. 3 is a view describing one process during manufacturing.

As shown in FIGS. 1(a) and 1(b), the probe 1 is used by being attached to the finger of a subject 2.

As shown in FIGS. 2(a) and 2(b), the probe 1 is configured to include a ring-shaped base material 70 and a flexible film 10 fixed to the inner circumferential surface of the ring-shaped base material 70. The ring-shaped base material 70 is partially cut out in the circumferential direction, has a C-shaped cross section, and is flexible.

On the films 10 at positions facing each other across the finger of the ring-shaped base material 70, semiconductor light-emitting elements 21a and 21b are mounted on one film and a semiconductor light-receiving element 31 on the other film.

The emission wavelength of the semiconductor light-emitting element 21a is red light, and the emission wavelength of the semiconductor light-emitting element 21b is infrared light. The semiconductor light-emitting elements 21a and 21b emit light toward the finger of the subject 2, and the semiconductor light-receiving element 31 receives the light that is emitted from the semiconductor light-emitting elements and passes through the finger of the subject 2.

Accordingly, the pulse oximeter connected to the probe 1 can calculate the ratio between the intensity of red light transmitted through the finger of the subject 2 and the intensity of infrared light transmitted through the finger, and calculate the arterial blood oxygen saturation (SpO₂), which indicates the percentage of hemoglobin in the arterial blood in the finger of the subject 2 that is bound to oxygen.

Here, both the semiconductor light-emitting elements 21a and 21b and the semiconductor light-receiving element 31 are bare chips including a semiconductor layer and a pair of electrodes provided on the semiconductor layer. The bare chip size of the semiconductor light-emitting elements 21a and 21b is about 300 μm square and 200 μm thick, and the bare chip size of the semiconductor light-receiving element 31 is about 3 mm square and 200 μm thick, both of which are small.

By using bare chips as the semiconductor light-emitting elements 21a and 21b and the semiconductor light-receiving element 31, the height (thickness) can be reduced to about 200 μm or less. Therefore, the semiconductor light-emitting elements 21a and 21b and the semiconductor light-receiving element 31 do not press against the skin of the subject 2.

The bare-chip semiconductor light-emitting elements 21a and 21b are mounted on the surface of the film 10 on the finger side of the subject 2. The light-emitting surfaces 44 the semiconductor light-emitting elements 21a and 21b are opposite to the film 10, that is, the surfaces on the finger side of the subject 2.

Meanwhile, the semiconductor light-receiving element 31 is mounted on the surface of the film 10 on the ring-shaped base material 70 side. The light-receiving surface of the semiconductor light-receiving element 31 is the surface on the film 10 side.

In this manner, by mounting the semiconductor light-receiving element 31, which has a larger bare chip size than the semiconductor light-emitting elements 21a and 21b, on the surface of the film 10 opposite the finger of the subject 2, the semiconductor light-receiving element 31 can be further prevented from pressing against the skin of the subject 2.

In addition, the semiconductor light-emitting elements 21a and 21b, which have small bare chip sizes, are mounted on the surface of the film 10 on the finger side of the subject 2, and thus the light emitted from the semiconductor light-emitting elements 21a and 21b can be emitted toward the finger without passing through the film 10 and being attenuated.

At least the region of the film 10 on which the semiconductor light-receiving element 31 is mounted has a property of transmitting light emitted by the semiconductor light-emitting elements 21a and 21b. As a result, the semiconductor light-receiving element 31 receives light that is emitted from the semiconductor light-emitting element and transmitted through the finger and the film 10.

The region of the film 10 on which the semiconductor light-emitting elements 21a and 21b are mounted, or the region on which the semiconductor light-receiving element 31 is mounted, may be folded to form a double layer. As a result, even when the semiconductor light-emitting elements 21a and 21b and the semiconductor light-receiving element 31 are formed on the same side of the film 10 during manufacturing, by folding the film 10, the semiconductor light-emitting elements 21a and 21b can be mounted on the surface of the film 10 on the finger side of the subject 2, and the semiconductor light-receiving element 31 can be mounted on the surface of the film 10 on the ring-shaped base material 70 side. Accordingly, it is possible to simplify the manufacturing process.

In Embodiment 1, as shown in FIGS. 2(a) and 2(b), the film 10 in the region where the semiconductor light-emitting elements 21a and 21b are mounted is folded in two.

A pair of electrodes of the semiconductor light-emitting elements 21a and 21b are directly bonded to wiring 11a provided on the surface of the film 10. A pair of electrodes of the semiconductor light-receiving element 31 are directly bonded to wiring 11b provided on the surface of the film 10.

The bonding material for bonding the electrodes of the bare-chip semiconductor light-emitting elements 21a and 21b and the semiconductor light-receiving element 31 onto the wiring 11a or 11b of the film 10 is preferably a sintered body of metal particles. By using a sintered body of metal particles, it is possible to sinter at a temperature that does not damage the flexible film 10.

In addition, the surroundings of the semiconductor light-emitting elements 21a and 21b and the semiconductor light-receiving element 31 are each sealed with a transparent sealing material 22 that transmits light emitted by the semiconductor light-emitting element.

The side surfaces of the sealing material 22 that seals the semiconductor light-emitting elements 21a and 21b are covered with a light-reflective resin 33.

The surrounding of the sealing material 22 that seals the semiconductor light-receiving element 31 is covered with the light-reflective resin 33. That is, the surfaces of the sealing material 22 other than the surface in contact with the film 10 (the side surfaces and the surface on the base material 70 side) are covered with the light-reflective resin 33.

Accordingly, among the light emitted from the semiconductor light-emitting elements 21a and 21b, the light that travels in the lateral direction is reflected by a light-reflective resin 23 and directed toward the subject 2, thereby increasing the amount of light emitted to the subject 2.

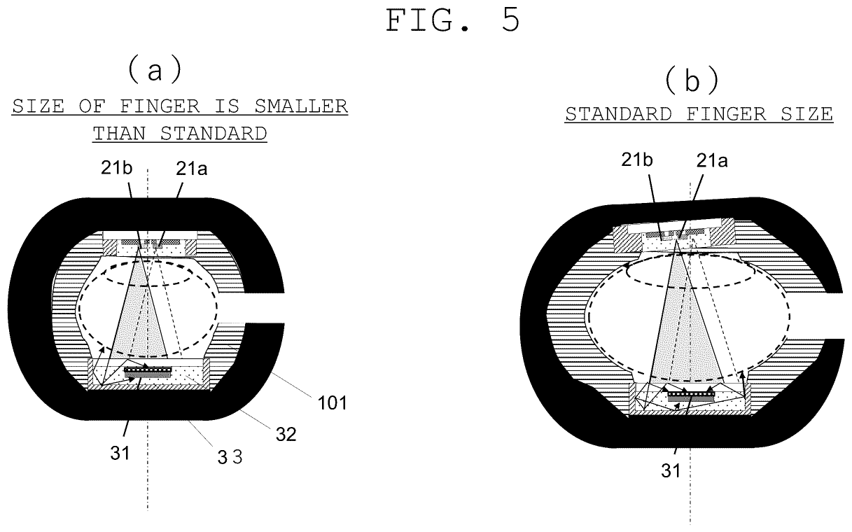

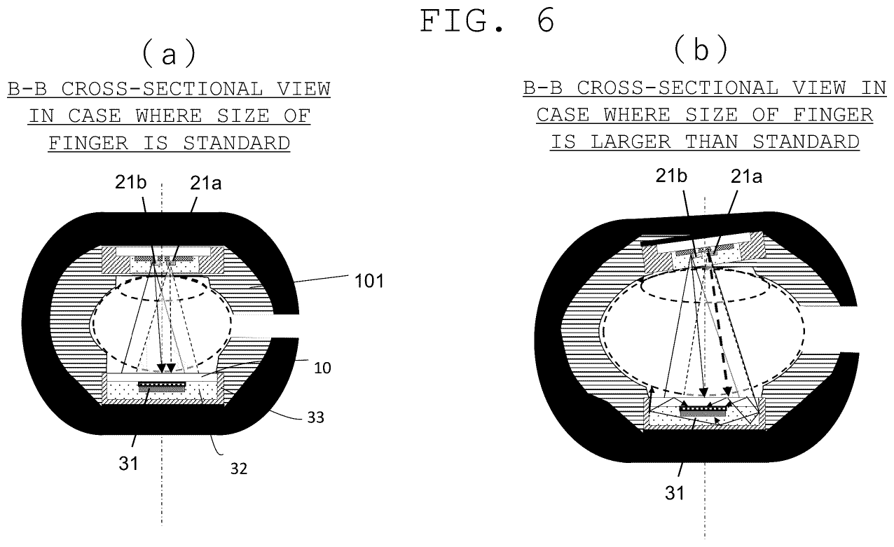

Furthermore, among the light that passes through the subject 2 and is directed toward the semiconductor light-receiving element 31, the light that is not directly made incident on the light-receiving surface of the semiconductor light-receiving element 31 is reflected by the light-reflective resin 33 around a transparent sealing material 32 as shown in FIGS. 5 and 6 and returned to the film 10 side, and is reflected at the interface between the film 10 and the air to be incident on the light-receiving surface of the semiconductor light-receiving element 31, thereby improving the light receiving efficiency.

Therefore, it is desirable that the refractive indexes of the transparent sealing material 32 and the film 10 are matched, or that the refractive index of the film 10 is greater than the refractive index of the sealing material 32. The light reflected by the light-reflective resin 33 is incident on the film 10 from the transparent sealing material 32, and is reflected by the interface between the film 10 and the air, and accordingly, the light is made incident on the light-receiving surface of the semiconductor light-receiving element 31.

The interface between the sealing material and the light-reflective resin may be formed such that a part of the interface is inclined with respect to the main plane of the film 10. Accordingly, it becomes possible to control the direction of light such that the light is incident on the light-receiving surface of the semiconductor light-receiving element 31, thereby further improving the light receiving efficiency of the semiconductor light-receiving element 31.

In addition, in the ring-shaped probe of the present embodiment, power supply wiring 50a for supplying power to the semiconductor light-emitting elements 21a and 21b is connected to the wiring 11a. Signal extraction wiring 50b for extracting the output signal of the semiconductor light-receiving element 31 is connected to the wiring 11b. The power supply wiring 50a and the signal extraction wiring 50b are partially provided in parallel on the film 10. The power supply wiring 50a and the signal extraction wiring 50b are collectively referred to as power supply/signal extraction wiring 50. A connection terminal 60 is attached to the tip of the power supply/signal extraction wiring 50.

As described above, in the probe 1 of Embodiment 1, bare chips are used as the semiconductor light-emitting elements 21a and 21b and the semiconductor light-receiving element 31, and accordingly, the semiconductor light-emitting elements 21a and 21b and the semiconductor light-receiving element 31 are configured not to press against the skin of the subject 2.

Moreover, the bare-chip semiconductor light-emitting elements 21a and 21b can directly conduct the generated heat to the wiring 11a and can dissipate the heat while further conducting the heat along the wiring 11a and the wiring 50. That is, the bare-chip semiconductor light-emitting elements 21a and 21b do not have a package substrate, cavity, or the like between the wiring 11a and the semiconductor light-emitting elements 21a and 21b, and therefore there is no member that would act as a thermal conduction bottleneck or no member that could retain heat. Therefore, compared to a packaged LED, the bare-chip semiconductor light-emitting elements 21a and 21b have superior heat dissipation characteristics and can dissipate heat efficiently from the wiring 11a. Accordingly, it is possible to suppress an increase in temperature of the semiconductor light-emitting elements 21a and 21b.

In addition, since the film 10 is a C-shaped ring type, the semiconductor light-emitting elements 21a and 21b and the semiconductor light-receiving element 31 can be pre-mounted at positions facing each other across the space in which the subject 2 is to be disposed. Therefore, the attachment of the probe 1 of the present embodiment to the subject 2 can be completed simply by fitting the probe 1 to the finger of the subject 2, and there is no need to align the semiconductor light-emitting elements 21a and 21b and the semiconductor light-receiving element 31. Accordingly, the burden on medical staff can be reduced.

In addition, by covering the side surfaces Of the sealing material 22 that seals the semiconductor light emitting elements 21a and 21b with the light-reflective resin 23 and covering the surrounding of the sealing material 32 that seals the semiconductor light-receiving element 31 with the light-reflective resin 33, the light-receiving efficiency of the semiconductor light-receiving element 31 can be improved.

In addition, it is desirable that the ring-shaped base material 70 has light-shielding properties. Accordingly, it is possible to prevent light leaking from the side surface of the light-reflective resin 33 of a light-emitting section 20 from propagating through the ring-shaped base material 70 and reaching a light-receiving section 30.

It is desirable that the transparent sealing materials 22 and 32, the light-reflective resins 23 and 33, and the ring-shaped base material 70 are all made of elastic materials.

It is desirable to dispose a protective sealing material 101 to smoothly fill the step between the transparent sealing material 22 and the light-reflective resin 23 around the semiconductor light-emitting elements 21a and 21b, and the film 10. By providing the protective sealing material 101, the inner circumferential surface of the probe for a pulse oximeter 1 can be molded even more smoothly. However, the protective sealing material 101 should not adhere to the surface (light-emitting surface) of the transparent sealing material 22 around the semiconductor light-emitting elements 21a and 21b. The protective sealing material 101 desirably has a light-shielding property, and can be formed of, for example, a silicone resin having a light-shielding property. By using the light-shielding protective sealing material 101, it is possible to prevent light emitted by the semiconductor light-emitting elements 21a and 21b and leaking out from the side surface of the light-reflective resin 23 from propagating through the protective sealing material 101 and reaching the semiconductor light-receiving element 31.

The transparent sealing materials 22 and 32, the light-reflective resins 23 and 33, and the protective sealing material 101 may be made of elastic materials. The light-reflective resins 23 and 33 and the protective sealing material 101 may be made of the same material that has light-reflective and light-shielding properties.

### During Use of Probe 1

When using the probe 1, the ring-shaped probe 1 is fitted to the finger of the subject 2, the terminal 60 is connected to the pulse oximeter device, and power is supplied to the semiconductor light-emitting elements 21a and 21b via the power supply/signal extraction wiring 50 and the wiring 11a or 11b. The red light and infrared light emitted from the semiconductor light-emitting elements 21a and 21b pass through the finger and are received by the semiconductor light-receiving element 31. The output of the semiconductor light-receiving element 31 is input to the pulse oximeter via the wiring 11b and the power supply/signal extraction wiring 50. The pulse oximeter calculates and displays the ratio of oxyhemoglobin (SpO₂) among the hemoglobin in the blood based on the output of the semiconductor light-receiving element 31, using the intensity ratio of red light and infrared light, or the like.

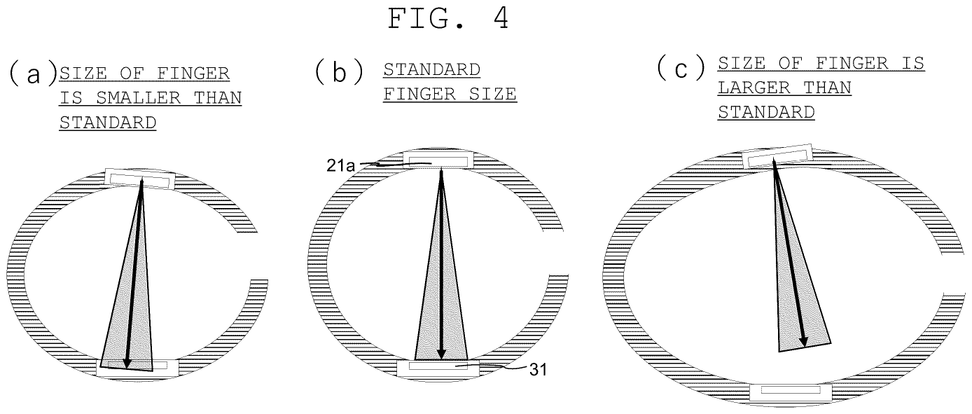

At this time, since the flexible ring-shaped base material 70 is C-shaped, the internal stress is generated that causes the cut-out portion of the C-shape to shrink in a narrowing direction. As shown in FIG. 4(b), when the ring-shaped probe 1 is attached to the finger of the subject 2 having the same size as a standard finger size assumed when designing the positions where the semiconductor light-emitting elements 21a and 21b and the semiconductor light-receiving element 31 face each other, the semiconductor light-emitting elements 21a and 21b and the semiconductor light-receiving element 31 face each other. However, when the size of the finger of the subject 2 is smaller than the standard finger size (FIG. 4(a)) or larger than the standard finger size (FIG. 4(c)), the optical axes Of the semiconductor light-emitting elements 21a and 21b are tilted with respect to the optical axis of the semiconductor light-receiving element 31, and the amount of light directly incident on the light-receiving surface of the semiconductor light-receiving element 31 is reduced compared to the case of FIG. 4(b).

However, in Embodiment 1, as shown in FIG. 5(a), the semiconductor light-receiving element 31 is bonded to the wiring 11b in a state where the transparent sealing material 32 is sandwiched between the semiconductor light-receiving element 31 and the film 10. Therefore, as shown in FIG. 5(b), when the size of the finger is larger than the standard, the optical axes of the semiconductor light-emitting elements 21a and 21b are tilted with respect to the optical axis of the semiconductor light-receiving element 31, and even when the amount of light directly incident on the light-receiving surface Of the semiconductor light-receiving element 31 is reduced, the light diffusely reflected by the light-reflective resin 33 is made incident on the transparent film and is totally reflected at the interface between the transparent film and the air, and is directed toward the light-receiving surface of the semiconductor light-receiving element 31. The transparent sealing material 32 is formed between the semiconductor light-receiving element 31 and the film 10, and by increasing the thickness of the wiring 11b to which the electrode pad of the semiconductor light-receiving element 31 is connected (or by increasing the thickness of only the electrode pad of the semiconductor light-receiving element 31), the distance (thickness) of the sealing material 32 between the back surface of the transparent film 10 and the semiconductor light-receiving element 31 can be increased. As a result, the distance can be ensured between the surface of the transparent film 10 and the semiconductor light-receiving element 31. That is, the thickness of the wiring 11b and/or the electrode pad serves as a thickness adjustment section that adjusts the distance (thickness) of the sealing material between the surface of the transparent film 10 and the semiconductor light-receiving element 31. As a result, the totally reflected light is directed toward the center of the semiconductor light-receiving element 31, making it possible to receive the light reliably. Even when the size of the finger is larger than the standard size and the amount of light incident on the light-receiving surface of the semiconductor light-receiving element 31 is reduced, the reduction can be compensated by the totally reflected light, thereby enabling highly accurate detection while suppressing the reduction in the amount of received light.

Furthermore, the semiconductor light-receiving element 31 has a light-receiving surface larger than the light-emitting surfaces of the semiconductor light-emitting elements 21a and 21b, thereby improving the efficiency of capturing the totally reflected light.

In addition, in the present embodiment, two semiconductor light-emitting elements 21a and 21b are used, and when the size of the finger is larger than the standard size (FIG. 6(a)), the optical axes of the two semiconductor light-emitting elements 21a and 21b are tilted with respect to the optical axis of the semiconductor light-receiving element 31 (FIG. 6(b)). The light emitted from the semiconductor light-emitting element 21b positioned at the back side of the C-shaped base material 70 is largely incident directly on the semiconductor light-receiving element 31 as shown in FIG. 6(b), and the received light intensity is unlikely to decrease. Meanwhile, the light emitted from the semiconductor light-emitting element 21a positioned on the cut-out side of the C-shaped base material 70 has a high proportion of light that deviates from the light-receiving surface of the semiconductor light-receiving element 31. However, light that misses the light-receiving surface is scattered by the light-reflective resin 33 (white resin), passes through the sealing material 32 and is made incident on the transparent film 10, is totally reflected at the interface between the transparent film 10 and the air, and can be made incident on the light-receiving surface of the semiconductor light-receiving element 31. Thus, the reduction in the amount of received light can be compensated.

Since the transparent film 10 is thin, the transparent sealing material 32 is formed between the semiconductor light-receiving element 31 and the transparent film such that the light totally reflected by the film 10 can reach the semiconductor light-receiving element 31, and the height is ensured such that the light can reach the semiconductor light-receiving element 31.

### Manufacturing Method

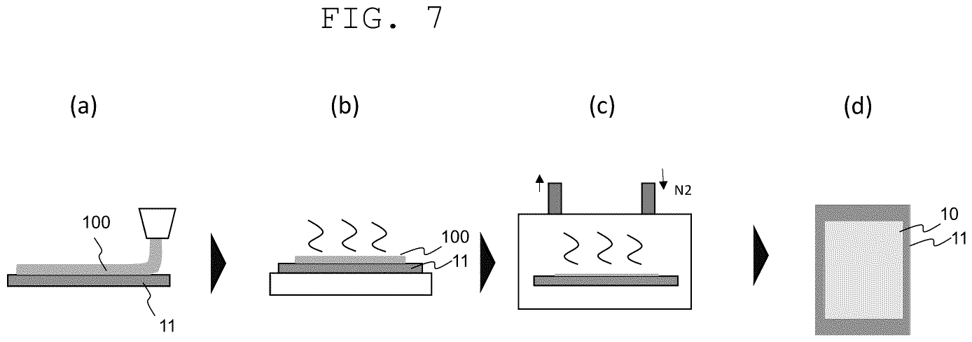

The structure of the probe for a pulse oximeter 1 according to Embodiment 1 will be described with reference to FIGS. 7 to 11.

### Process for Producing Film 10 With Wirings 11a and 11b

As shown in FIG. 7(a), an uncured transparent polyimide layer 100 is applied onto a copper foil 11 (having a thickness of, for example, 12 μm) with a die coater to a certain thickness (for example, 70 μm).

As shown in FIG. 7(b), the copper foil 11 with an uncured polyimide layer 100 is heated using a hot plate or the like at a predetermined temperature and for a predetermined time (for example, 100° C. for 10 minutes) to perform preliminary drying. At this time, a hole may be provided in the hot plate, and the copper foil 11 with the polyimide layer 100 may be heated while being adsorbed to the hot plate. If necessary, a frame may be placed on the copper foil 11 with the polyimide layer 100 to press it down.

As shown in FIG. 7(c), the copper foil 11 with the polyimide layer 100 is heated in an oven in a nitrogen atmosphere at a predetermined temperature for a predetermined time (for example, 260° C. for 60 minutes) to perform main drying. As a result, a transparent film 10 (transparent polyimide: thickness 35 μm) integrated with the copper foil 11 is formed (FIG. 7(d)).

Next, the copper foil on the film 10 in FIG. 7(d) is processed by etching to form the pattern of the wiring 11a on which the semiconductor light-emitting elements 21a and 21b are mounted, the pattern of the wiring 11b on which the semiconductor light-receiving element 31 is mounted, and the pattern of the power supply/signal extraction wiring 50. After processing, a gold plating layer is formed on the copper foil surface by electroless plating. Specifically, the wiring pattern shown in FIG. 3 is formed. All of the wirings 11a, 11b, and 50 are formed by processing the copper foil on the same surface of the film 10.

### Process for Mounting Semiconductor Light-Emitting Elements 21a and 21b

The semiconductor light-emitting elements 21a and 21b are mounted on the wiring 11a on the film 10. The semiconductor light-emitting elements 21a and 21b are of a flip chip type, in which the electrodes are on the same surface side and light is emitted from the surface opposite to the electrodes.

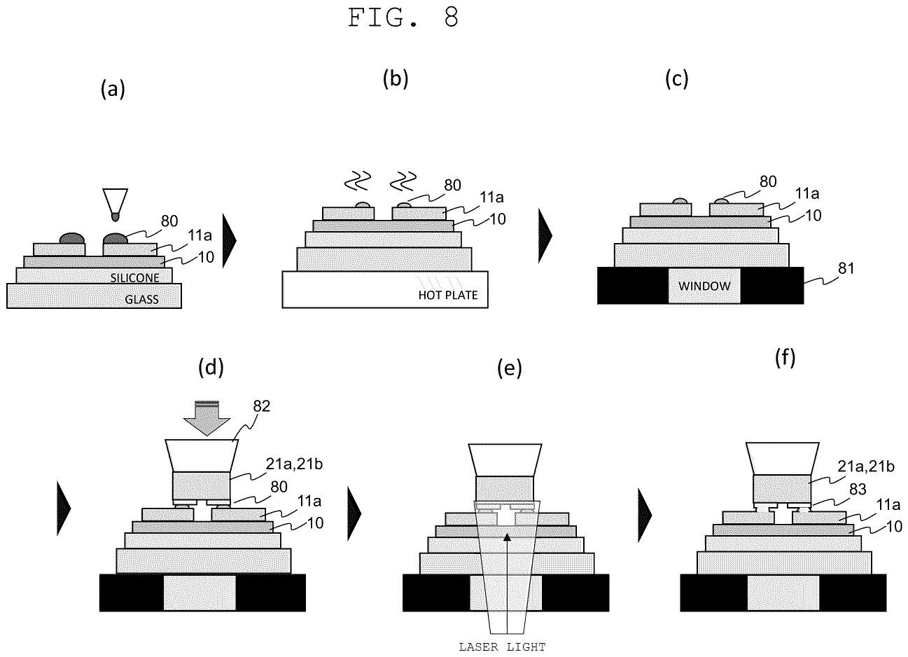

Specifically, as shown in FIG. 8(a), Au ink 80, in which gold particles with an average particle diameter of 30 nm are dispersed in a solvent (glycerin) at a concentration of 82 wt %, is prepared and applied with a dispenser to the parts of the wiring 11a where the semiconductor light-emitting elements 21a and 21b are to be mounted. At this time, it is preferable that the film 10 is mounted on a silicon substrate, and further on a glass substrate.

Next, as shown in FIG. 8(b), the Au ink 80 is dried by heating on a hot plate at a predetermined temperature for a predetermined time (for example, 50°C. for 45 to 60 minutes).

As shown in FIG. 8(c), the film 10 is set in an optical bonding device 81, and as shown in FIG. 8(d), the electrodes of the semiconductor light-emitting elements 21a and 21b are mounted on the Au ink 80, and the semiconductor light-emitting elements 21a and 21b are pressed against the wiring 11a with a predetermined pressure using a load collet 82.

As shown in FIG. 8(e), blue laser light having a predetermined beam diameter is irradiated from the bottom surface of the film 10 onto the region of the wiring 11a where the Au ink 80 is mounted. The region of the wiring 11a that is irradiated with the blue laser light absorbs the blue laser light and is heated, and this heat is conducted to the Au ink 80, heating the Au ink 80. The gold particles in the Au ink 80 have a small average particle size of 30 nm, and are sintered at a temperature lower than the melting point of Au to form a sintered body, forming a bonding material 83 that bonds the wiring 11a to the electrodes of the semiconductor light-emitting elements 21a and 21b.

At this time, since the film 10 is transparent, it does not absorb the blue laser light and is not directly heated. The heat of the wiring 11a heated by the blue laser light is conducted to the region of the wiring 11a that is not irradiated with the blue laser light, and is rapidly dissipated. Therefore, without damaging the semiconductor light-emitting elements 21a and 21b or the resin film 10, the semiconductor light-emitting elements 21a and 21b can be mounted on the wiring 11a by optical bonding using the bonding material 83 formed of a gold particle sintered body.

### Process for Mounting Semiconductor Light-Receiving Element 31

The semiconductor light-receiving element 31 is a flip chip type photodiode in which a pair of electrodes are on the same surface side, and one having a light-receiving surface between the pair of electrodes is used.

The semiconductor light-receiving element 31 is mounted on the wiring 11b of the film 10. The mounting method may be the same optical bonding method as in FIGS. 8(a) to 8(f), or, since the semiconductor light-receiving element 31 is larger in size than the semiconductor light-emitting elements 21a and 21b, the semiconductor light-receiving element 31 may be bonded by soldering.

### Sealing and Assembly Process

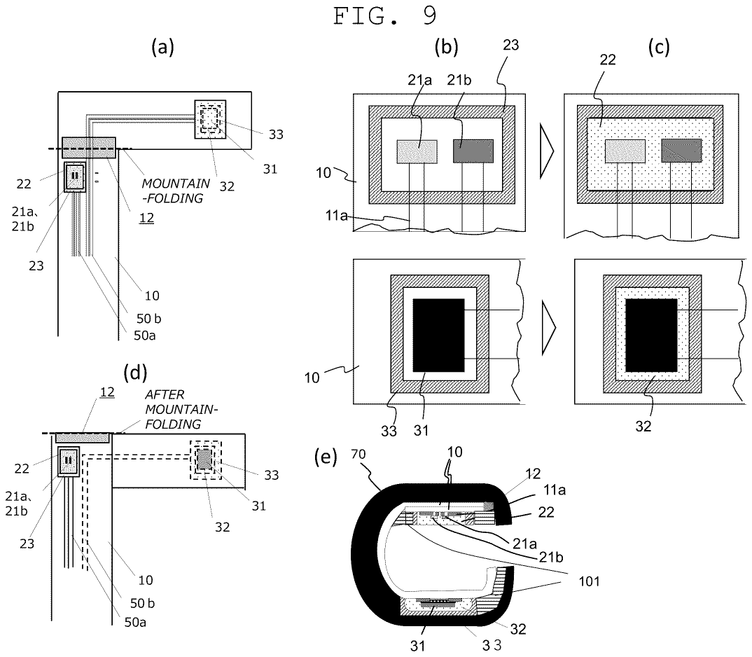

As shown in FIG. 9(a), the film 10, on which the wiring 11a on which the semiconductor light-emitting elements 21a and 21b are mounted, the wiring 11b on which the semiconductor light-receiving element 31 is mounted, and the power supply/signal extraction wiring 50 connected thereto are formed, is cut out into the shape shown in FIGS. 3 and 9(a).

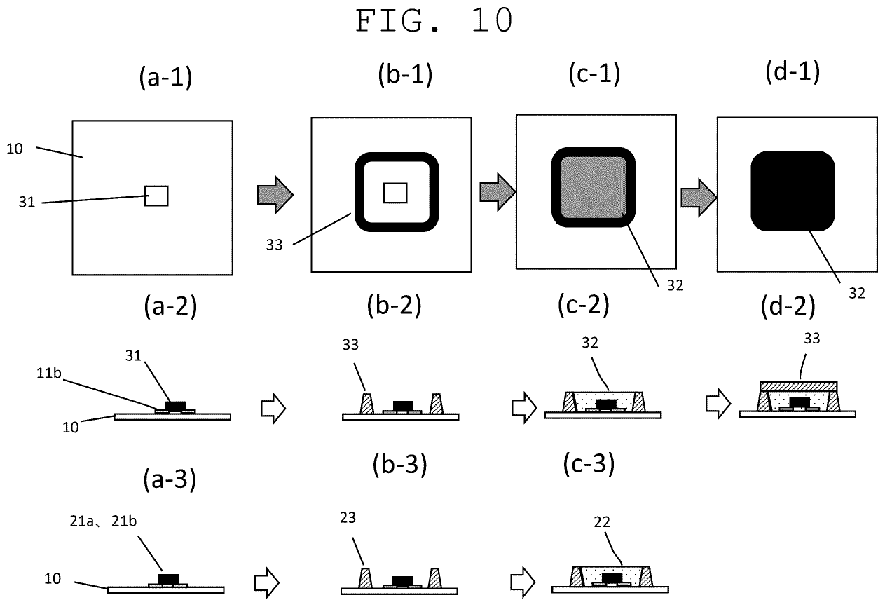

An uncured white resin having titanium oxide, zinc oxide or aluminum oxide dispersed in the silicone resin is prepared, and is applied in a frame shape surrounding the semiconductor light-emitting elements 21a and 21b and the semiconductor light-receiving element 31 (FIGS. 10(a-1) to 10(a-3)) at positions a predetermined distance away from the semiconductor light-emitting elements 21a and 21b and the semiconductor light-receiving element 31, respectively, to form the uncured light-reflective resins 23 and 33 (FIG. 9(b) and FIGS. 10(b-1) to 10(b-3)). The frame-shaped light-reflective resins 23 and 33 are formed to have a height that is designed in advance and is higher than the heights of the semiconductor light-emitting elements 21a and 21b and the semiconductor light-receiving element 31.

As shown in FIG. 9(c) and FIGS. 10(c-1) to 10(c-3), transparent silicone resin is injected into the frame-shaped light-reflective resin 23, 33 that has been formed, to form the uncured transparent sealing materials 22 and 32 that seal the surrounding of the semiconductor light-receiving element 31 and the semiconductor light-emitting elements 21a and 21b. Thereafter, the film 10 is heated at a predetermined temperature for a predetermined p time (for example, 150° C. for 4 hours) to harden or temporarily harden the light-reflective resins 23 and 33 and the transparent sealing materials 22 and 32. As a result, the transparent sealing materials 22 and 32 were formed with a thickness of 30 μm in the optical axis direction.

As shown in FIGS. 10(d-1) and 10(d-2), the transparent sealing material 32 that seals the semiconductor light-receiving element 31 is covered with uncured white resin, and heated at a predetermined temperature for a predetermined time to form the light-reflective resin 33 that covers the surrounding of the transparent sealing material 32.

As shown in FIG. 9(d), an adhesive layer 12 is formed by applying silicone resin or polyimide varnish to predetermined mountain-folded area of the film 10 on which the semiconductor light-emitting elements 21a and 21b and the semiconductor light-receiving element 31 are mounted.

By mountain-folding the film 10, the orientations of the semiconductor light-emitting elements 21a and 21b and the semiconductor light-receiving element 31 are oriented as shown in FIGS. 2(a) and 2(b). As a result, the film 10 becomes double in the regions where the semiconductor light-emitting elements 21a and 21b are mounted.

The C-shaped ring-shaped base material 70 is prepared in advance from a flexible resin such as acrylic containing a light-shielding pigment. The film in the shape of FIG. 9(d) adheres to the inner circumferential surface of the C-shaped ring-shaped base material 70 with an adhesive (FIG. 9(e)).

Furthermore, the protective sealing material 101 is applied and cured to smoothly fill the step between the transparent sealing material 22 and the light-reflective resin 23 around the semiconductor light-emitting elements 21a and 21b, and the film 10. As a result, the inner circumferential surface of the probe for a pulse oximeter 1 is formed smoothly. The protective sealing material 101 may be filled and cured using a mold to fill the step between the light-reflective resin 23 and the film 10. In either case, the protective sealing material 101 should not adhere to the surface (light-emitting surface) of the transparent sealing material 22 around the semiconductor light-emitting elements 21a and 21b.

The transparent sealing materials 22 and 32, the light-reflective resins 23 and 33, and the protective sealing material 101 may be made of elastic materials. The light-reflective resins 23 and 33 and the protective sealing material 101 may be made of the same material that has light-reflective and light-shielding properties.

In addition, in FIG. 9(e), the sizes of the minute semiconductor light-emitting elements 21a and 21b and the semiconductor light-receiving element 31 are drawn larger than their actual sizes in order to make the structure easier to understand visually.

Finally, the connection terminal 60 is connected to the tip of the power supply/signal extraction wiring 50 that is pulled out from the ring-shaped film 10.

Through the above steps, the probe for a pulse oximeter 1 of Embodiment 1 is completed.

In the above manufacturing method, the C-shaped ring-shaped base material 70 is prepared in advance; however, after placing the film having the shape shown in FIG. 9(d) into a mold, the ring-shaped base material 70 may be molded by pouring resin into the mold.

Specifically, the film 10 having the shape shown in FIG. 9(d) is set along a mold in which a C-shaped ring-shaped recess portion is formed. At this time, the power supply/signal extraction wiring 50 and the part of the film 10 on which the power supply/signal extraction wiring 50 is mounted are set to be pulled out from the mold. A silicone resin or the like having light-shielding properties is filled around the film 10. At this time, a mold is formed on the inner circumferential surfaces of the light-emitting section 20 and the light-receiving section 30 such that the light-shielding ring-shaped base material 70 does not adhere thereto.

The ring-shaped base material 70 in the mold is hardened under pressure at a predetermined temperature for a predetermined time (for example, 150° C. for 4 hours). Accordingly, the light-shielding ring-shaped base material 70 is formed with the film 10 of FIG. 9(d) adhered to the inner circumferential surface, and a by removing it from the mold, the probe 1 with the structure of FIG. 9(e) can be manufactured.

### Embodiment 2

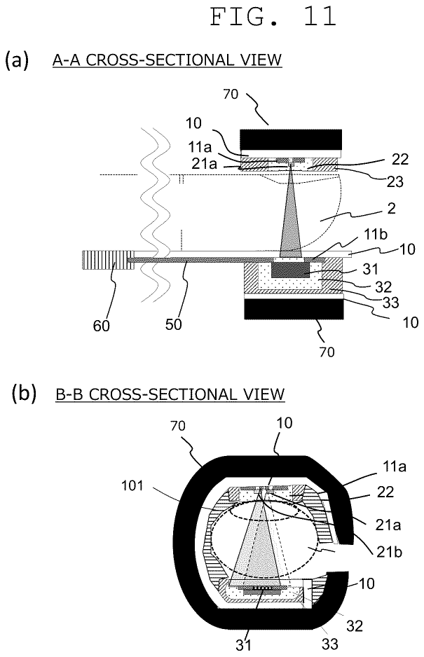

The probe for a pulse oximeter 1 according to Embodiment 2 will be described with reference to FIGS. 11 to 13.

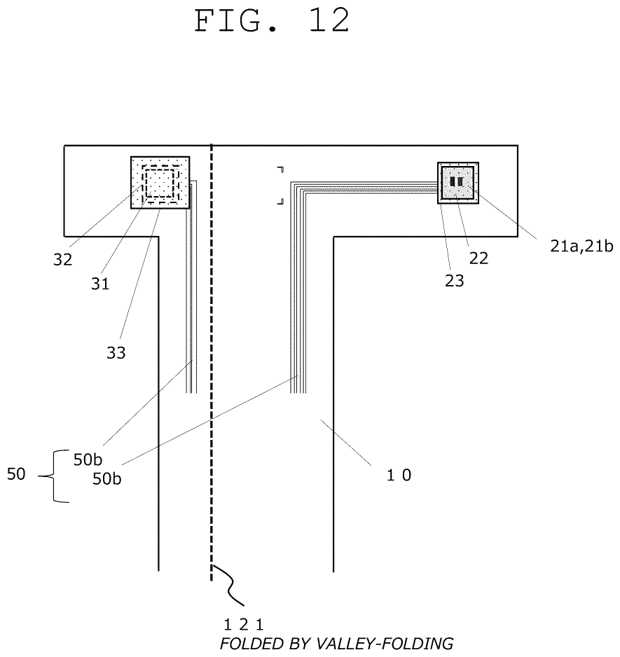

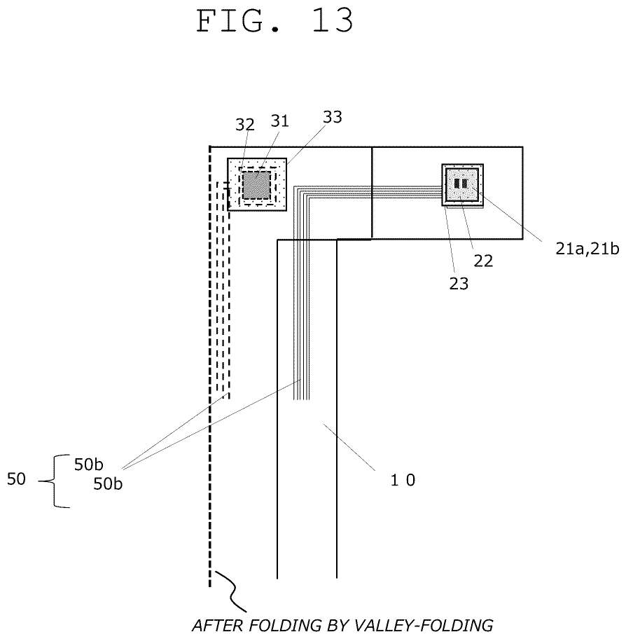

FIGS. 11(a) and 11(b) are sectional views taken along lines A-A and B-B of the probe 1. FIGS. 12 and 13 are views describing a process during manufacturing.

The probe in FIGS. 12(a) and 12(b) has a configuration similar to that of the probe in FIG. 2 of Embodiment 1, but differs from Embodiment 1 in that the film 10 is double-layered in the region where the semiconductor light-receiving element 31 is mounted. In addition, the semiconductor light-receiving element 31 and the transparent sealing material 32 and light-reflective resin 33 are disposed between the two layers of the film 10, and electrodes are bonded to the wiring 11b of the film 10 positioned on the finger side of the subject 2 of the double layered film 10.

The manufacturing method of the probe of Embodiment 2 is similar to the manufacturing method of the probe 1 of Embodiment 1, but in the process of FIG. 9(a) of Embodiment 1, when cutting out the film 10, the film 10 is cut into the shape shown in FIG. 12 and valley-folded at the position of the dotted line 121 to form the shape shown in FIG. 13, and the orientation of the light-receiving surface of the semiconductor light-receiving element 31 is reversed.

In the probe of Embodiment 2, the configuration and manufacturing process other than those described above are similar to those of Embodiment 1, and therefore description thereof will be omitted. Moreover, the operation of each part of the probe of Embodiment 2 is similar to that of the probe of Embodiment 1.

### Modification Example 1

In the probe for a pulse oximeter of Embodiments 1 and 2, modification examples of the interface shape between the transparent sealing material 32 that seals the surrounding of the semiconductor light-receiving element 31 and the light-reflective resin 33 that covers the surrounding thereof will be described.

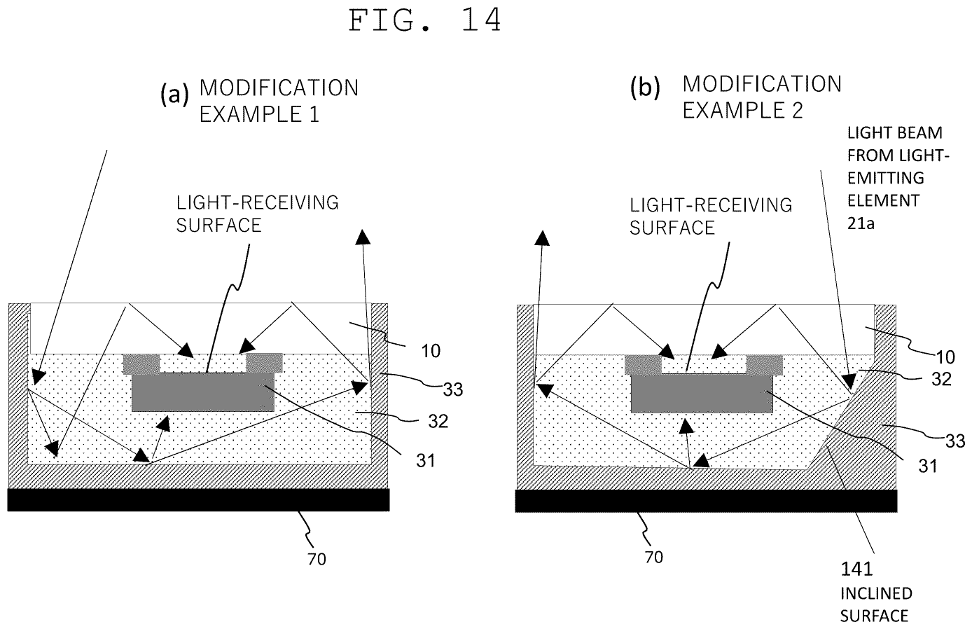

Modification Example 1 shown in FIG. 14(a) has a sealing material 32 with a rectangular parallelepiped shape, and the surrounding thereof is covered with the light-reflective resin 33 of uniform thickness. The refractive indexes of the sealing material 32 and the film 10 are made to match such that the light scattered and reflected by the light-reflective resin 33 can easily reach the light-receiving surface on the upper surface of the semiconductor light-receiving element 31. The refractive index and the thickness of the film 10 are designed such that, among the scattered reflected light incident on the film 10, the light that is totally reflected at the interface between the film 10 and the air is incident on the light-receiving surface on the upper surface of the semiconductor light-receiving element 31.

### Modification Example 2

Modification Example 2 shown in FIG. 14(b) is an example in which an inclined surface 141 is provided from the side surface of the cut-out side of the C-shaped base material 70 to the bottom surface at the interface between the transparent sealing material 32 and the light-reflective resin 33.

As shown in FIGS. 5(b) and 6(b), when the size of the finger is larger than the standard size, the range reached by the light from the semiconductor light-emitting element 21a shifts toward the cut-out part of the C-shaped base material 70. Therefore, by forming the part where the shifted light is irradiated into the inclined surface 141, the reflected light at the inclined surface can reach the interface between the film 10 and the air at an angle close to the total reflection angle, making it easier for the light to be incident on the light-receiving surface of the semiconductor light-receiving element 31.

### Modification Example 3

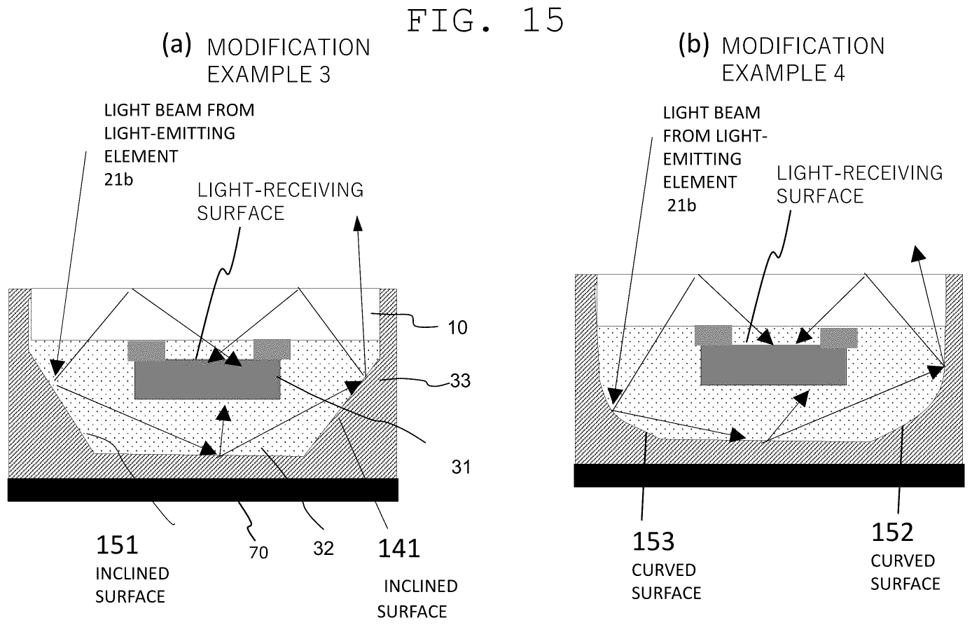

Modification Example 3 shown in FIG. 15(a) is an example in which, in addition to the inclined surface 141 of Modification Example 2, an inclined surface 151 is also provided from the side surface on the opposite side to the bottom surface.

Accordingly, when the size of the finger is smaller than the standard size as shown in FIG. 4(a), the light shifted to the back side of the C-shaped base material 70 can be reflected by the inclined surface 151.

Therefore, as in FIGS. 5(b) and 6(b), when the size of finger is larger than standard, the light with a shifted optical axis is reflected by the inclined surface 141, and when the size of the finger is smaller than standard as in FIG. 4(a), the light with a shifted optical axis is reflected by the inclined surface 151 and can reach the interface between the film 10 and the air at an angle close to the total reflection angle, making it easier for the light to be incident on the light-receiving surface of semiconductor light-receiving element 31.

### Modification Example 4

Modification Example 4 shown in FIG. 15(b) is an example in which the inclined surface 141 and the inclined surface 151 of Modification Example 3 are replaced with a curved surface 152 and a curved surface 153.

When the curved surfaces 152 and 153 (R-surfaces) are used instead of the inclined surfaces 141 and 151, the light incident on the sealing material 32 can reach the light-receiving surface, similarly to Modification Example 3.

### Modification Example 5

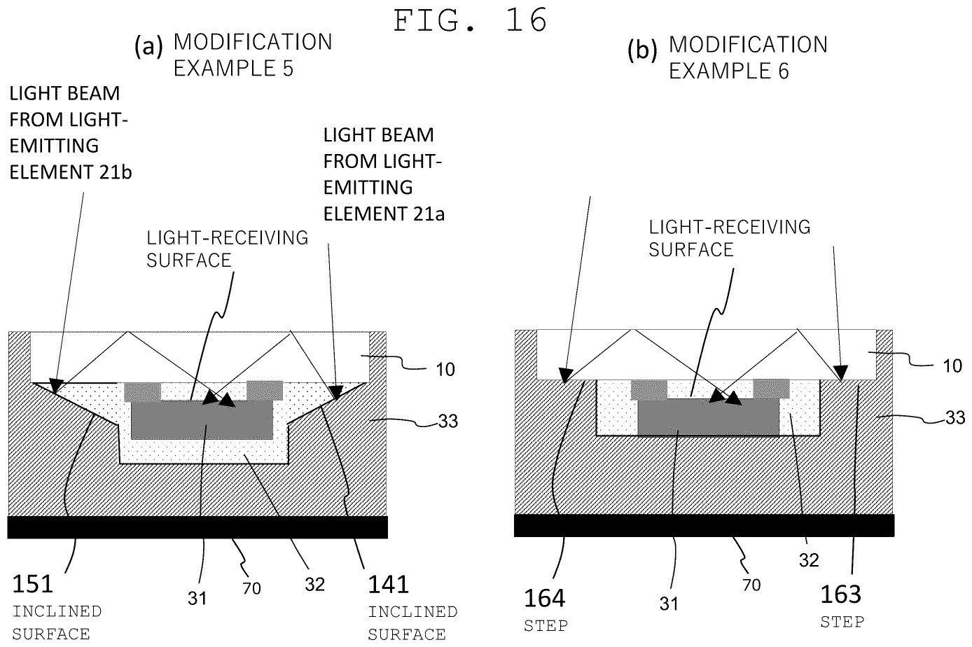

Modification Example 5 shown in FIG. 16(a) is an example in which the positions of the inclined surface 141 and the inclined surface 151 of Modification Example 3 are disposed closer to the film 10. The same effects and advantages as those of Modification Example 3 can be obtained.

### Modification Example 6

Modification Example 5 shown in FIG. 16(b) is an example in which steps 163 and 164 are provided in place of the inclined surface 141 and the inclined surface 151 of Modification Example 5. The same effects and advantages as those of Modification Example 5 can be obtained.

### Modification Example 7

In Modification Example 7, in the structures shown in Embodiments 1 and 2 and Modification Examples 1 to 6, a material having a higher refractive index than the film 10 is used as the transparent sealing material 32.

As a result, the light which is emitted by the semiconductor light-emitting elements 21a and 21b and passes through the film 10 on which the semiconductor light-receiving element 31 is mounted but is not directly made incident on the light-receiving surface of the semiconductor light-receiving element 31, is made incident on the transparent sealing material 32, is reflected by the light-reflective resin 33, and returns to the film 10 again. In this case, since the transparent sealing material 32 is made of a resin with a refractive index higher than the refractive index (=1.6) of the film 10 (for example, polyimide), no reflection occurs at the interface between the transparent sealing material 32 and the film 10, and the angle of incidence with respect to the normal to the film 10 becomes large. That is, since the light is refracted in a direction where the traveling direction becomes a shallower angle relative to the main plane of the film 10, it becomes more likely to undergo total reflection at the interface between the film 10 and the air, making it easier for the light to be incident on the light-receiving surface.

### Embodiment 3

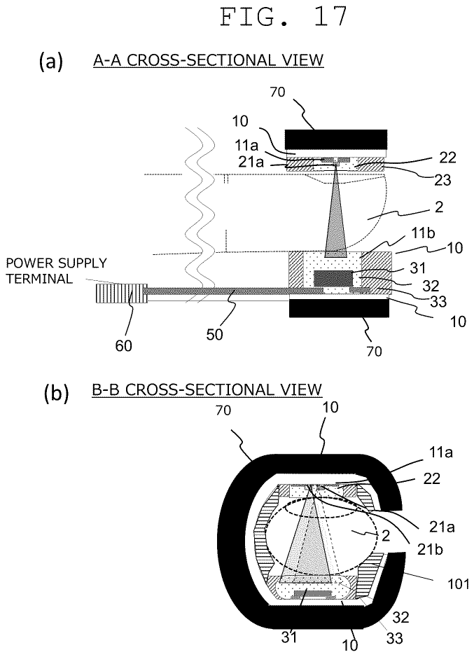

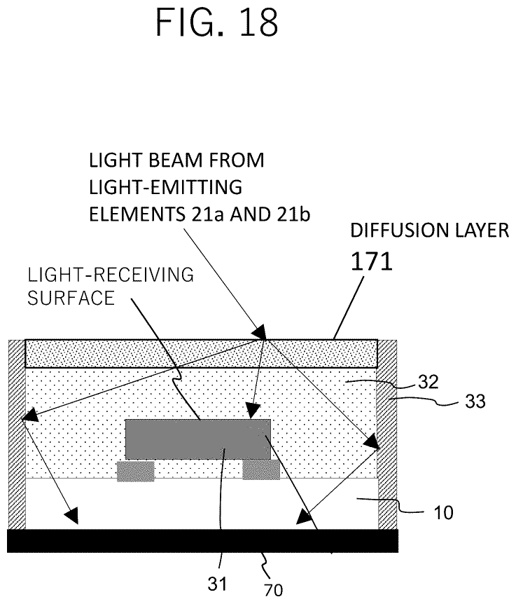

The probe of Embodiment 3 will be described with reference to FIGS. 17(a), 17(b), and 18.

In the Embodiments 1 and 2 and Modification Examples 1 to 6, the semiconductor light-receiving element 31 is disposed closer to the base material 70 than the film 10, but in Example 3, the semiconductor light-receiving element 31 is disposed closer to the finger of the subject 2 than the film 10. In this case, the light-receiving surface of the semiconductor light-receiving element 31 is also directed toward the finger of the subject 2.

As a result, the light emitted from the semiconductor light-emitting elements 21a and 21b passes through the finger of the subject 2, reaches the light-receiving surface of the light-receiving element 31 where the light is received.

In addition, as shown in FIG. 18, it is desirable to provide a diffusion layer 171 on the upper surface of the transparent sealing material 32. As a result, the light emitted from the semiconductor light-emitting elements 21a and 21b passes through the finger of the subject 2 and enters the diffusion layer 171. The light is diffused by the diffusion layer 171 and reaches the light-receiving surface of the semiconductor light-receiving element 31 where the light is received.

By arranging the diffusion layer 171 in this manner, even when the optical axis of the light emitted from the semiconductor light-emitting elements 21a and 21b is shifted when the size of the finger of the subject 2 is larger or smaller than standard, the light can be diffused by the diffusion layer 171 and reach the light-receiving surface.

### Modification Example 8

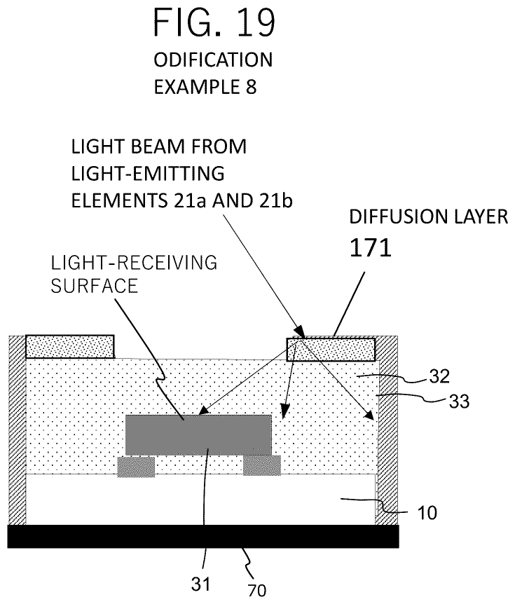

As a modification example of Embodiment 3, Modification Example 8 will be described with reference to FIG. 19.

Modification Example 8 of Embodiment 3 differs from Embodiment 3 in that, as shown in FIG. 19, the diffusion layer 171 is not provided in the region directly above the semiconductor light-receiving element 31, but is provided only on the upper surface of the sealing material 32 around the semiconductor light-receiving element 31.

In Modification Example 8, since the diffusion layer 171 is not provided in the region directly above the semiconductor light-receiving element 31, even when the size of the finger of the subject 2 is different from the standard size and the optical axes of the semiconductor light-emitting elements 21a and 21b are shifted, the light traveling straight toward the light-receiving surface does not pass through the diffusion layer 171, but passes through the sealing material 32 and can reach the light-receiving surface directly. As a result, the light traveling straight toward the light-receiving surface can be made incident on the light-receiving surface without being attenuated by the diffusion layer 171.

Meanwhile, the light that cannot reach the light-receiving surface directly due to the shift of the optical axis can be diffused by the diffusion layer 171, allowing a part of the light to reach the light-receiving surface.

Accordingly, it is possible to increase the amount of light that reaches the light-receiving surface of the semiconductor light-receiving element 31 as compared to the structure of Embodiment 3.

### Modification Example 9

As a modification example of Embodiment 3, Modification Example 9 will be described with reference to FIG. 19.

In the same manner as in Embodiment 3, Modification Example 9 has a configuration in which the semiconductor light-receiving element 31 is disposed closer to the finger of the subject 2 than the film 10, and the diffusion layer 171 is provided on the upper surface of the sealing material 32, but differs from Modification Example 8 in that the light-reflective resin 33 is also disposed between the film 10 and the base material 70. Another difference from Embodiment 3 is that the light-receiving surface of the semiconductor light-receiving element 31 is directed toward the film 10.

With such a configuration, in Modification Example 9, the light emitted from the semiconductor light-emitting elements 21a and 21b passes through the finger of the subject 2, is made incident on the diffusion layer 171, is diffused by the diffusion layer 171, and is further diffusely reflected by the light-reflective resin, and then reaches and is received by the light-receiving surface on the lower surface side of the semiconductor light-receiving element 31.

In the structure of Modification Example 9, the light-reflective resin 33 is disposed between the light-shielding base material 70 and the film 10, and thus the light is not absorbed by the light-shielding base material 70 and can efficiently reach the light-receiving surface.

### Modification Example 10

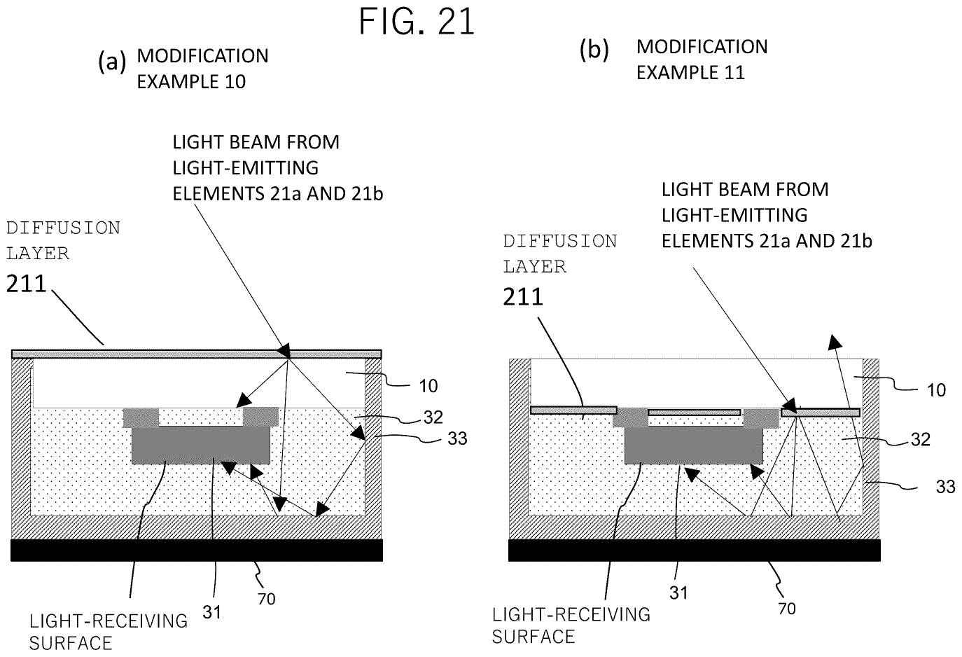

Here, as a modification example of the probe of Embodiment 1, an example in which a diffusion layer is provided will be described with reference to FIG. 21(a).

As shown in FIG. 21(a), the semiconductor light-receiving element 31 is disposed on the base material 70 side of the film 10, as in Embodiment 1, but the light-receiving surface of the semiconductor light-receiving element 31 is oriented toward the opposite side of the film 10.

The film 10 has a diffusion layer 211 mounted on the surface on the finger side of the subject 2.

In this configuration, the light emitted from the semiconductor light-emitting elements 21a and 21b passes through the finger of the subject, and first reaches the diffusion layer 211 where the light is diffused. The diffused light is further diffusely reflected one or more times by the light-reflective resin 33 and reaches the light-receiving surface of the semiconductor light-receiving element 31 where the light is received.

In the case of the configuration of FIG. 21(a), the diffusion layer 211 can be disposed by affixing a diffusion film or the like to the surface of the film 10.

### Modification Example 11

Modification Example 11 shown in FIG. 21(b) has a configuration in which the diffusion layer 211 of FIG. 21(a) is disposed between the base material 70 and the transparent sealing material 32.

In this configuration, similarly to Modification Example 10 of FIG. 21(a), the light emitted from the semiconductor light-emitting elements 21a and 21b passes through the finger of the subject, and first reaches the diffusion layer 211 where the light is diffused. The diffused light is further diffusely reflected one or more times by the light-reflective resin 33 and reaches the light-receiving surface of the semiconductor light-receiving element 31 where the light is received.

When manufacturing a probe having the diffusion layer 211 between the base material 70 and the transparent sealing material 32 as shown in FIG. 21(b), the diffusion layer 211 is formed during manufacturing in a process similar to the processes shown in FIGS. 10(a-2) to 10(d-2) of Embodiment 1.

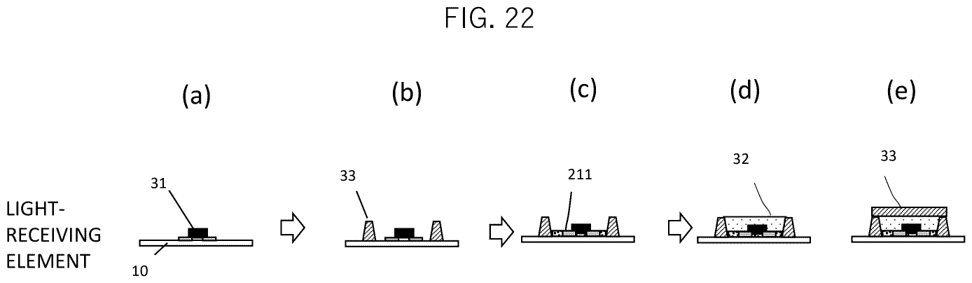

Specifically, an uncured white resin having titanium oxide, zinc oxide, or aluminum oxide dispersed in silicone resin is prepared, and the white resin is applied onto the film 10 at a predetermined distance from the semiconductor light-receiving element 31 (FIG. 22(a)) in a frame shape surrounding the semiconductor light-receiving element 31, to form the uncured light-reflective resin 33 (FIG. 22(b)). The height of the frame-shaped light-reflective resins 23 and 33 is greater than the height of the semiconductor light-receiving element 31.

Here, a transparent resin with diffuser particles dispersed therein is prepared and spread over the surface of the film 10 on the inner side of the frame-shaped light-reflective resin 33 to form the uncured diffusion layer 211 (FIG. 22(c).

As shown in FIG. 22(d), within the frame-shaped light-reflective resins 23 and 33, a transparent silicone resin is injected onto the diffusion layer 211 to form the uncured transparent sealing material 32 that seals the semiconductor light-receiving element 31. Thereafter, the film 10 is heated at a predetermined temperature for a predetermined time (for example, 150° C. for 4 hours) to harden or temporarily harden the light-reflective resins 23 and 33 and the transparent sealing materials 22 and 32. In this manner, the transparent sealing material 32 was formed.

As shown in FIG. 22(e), the transparent sealing material 32 is further covered with the uncured white resin to form the uncured light-reflective resin 23.

The transparent sealing material 32, the diffusion layer 211, and the light-reflective resin 33 are hardened by heating at a predetermined temperature for a predetermined time.

The other manufacturing processes are the same as those in Embodiment 1, and therefore the description thereof will be omitted.

### Modification Example 12

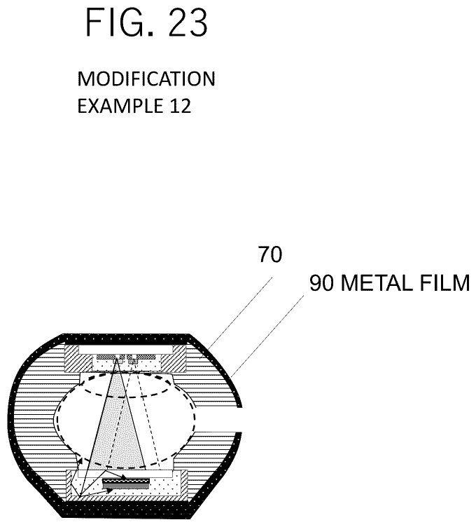

Modification Example 12 will be described with reference to FIG. 23. In the probe of Modification Example 12, a metal film 90 is disposed on the surface on the outer peripheral side of the ring-shaped base material 70 of the probe for a pulse oximeter according to Embodiments 1 to 3 or Modification Examples 1 to 11.

The metal film 90 can dissipate the heat generated by the light-emitting section 20, and therefore can improve the heat dissipation effect of the probe. In addition, the metal film 90 also acts as an electromagnetic shield, and can prevent external electromagnetic noise from entering the light-emitting section 20, the light-receiving section 30, and the wirings 11a and 11b.

The material of the metal film 90 may be a highly conductive metal such as copper or aluminum. The film thickness is preferably from 1 μm to 100 μm.

The metal film 90 can be formed by a vapor deposition method or plating after the formation process of the ring-shaped base material 70. Furthermore, when forming the ring-shaped base material 70 using a mold, a metal foil may be disposed in the mold to follow the outer circumferential portion of the ring-shaped base material 70 and cured simultaneously with the ring-shaped base material 70. In addition, a metal ring that has been separately produced in advance using metal processing technology may be attached or adhere to the outer side of the ring-shaped base material 70.

### Application to Products

The probe for a pulse oximeter of the present embodiment can be used not only for a pulse oximeter, but also as a light source unit for other wearable devices that optically acquire biological information, such as a smart ring.

### REFERENCE SIGNS LIST

1: probe for pulse oximeter
2: subject
10: film
10: base material
11: copper foil
11a: wiring
11b: wiring
12: adhesive layer
20: light-emitting section
21a: semiconductor light-emitting element
21b: semiconductor light-emitting element
22: sealing material
23: light-reflective resin
30: light-receiving section
31: semiconductor light-receiving element
32: sealing material
33: light-reflective resin
50: wiring
50a: power supply wiring
50b: signal extraction wiring
60: connection terminal
70: base material
80: ink
81: optical bonding device
82: load collet
83: bonding material
90: metal film
100: polyimide layer
101: protective sealing material
121: dotted line
141: inclined surface
151: inclined surface
152: curved surface
153: curved surface
163: step
171: diffusion layer
211: diffusion layer

1. A probe for a pulse oximeter comprising:
a ring-shaped base material to be attached to a finger of a subject;
a flexible film mounted on an inner circumferential surface of the ring-shaped base material; and
a semiconductor light-emitting element and a semiconductor light-receiving element mounted on the film at positions facing each other across the finger of the ring-shaped base material, wherein
the ring-shaped base material is partially cut out in a circumferential direction, has a C-shaped cross section, and is flexible,
each of the semiconductor light-emitting element and the semiconductor light-receiving element is a bare chip including a semiconductor layer and a pair of electrodes provided on the semiconductor layer, the pair of electrodes of the semiconductor light-emitting element and the semiconductor light-receiving element are directly bonded to wiring provided on a surface of the film,
surroundings of the semiconductor light-emitting element and the semiconductor light-receiving element are each sealed with a sealing material that transmits the light emitted by the semiconductor light-emitting element, and
the semiconductor light-emitting element emits light toward the finger, and the semiconductor light-receiving element receives light that is emitted from the semiconductor light-emitting element and passes through the finger.

2. The probe for a pulse oximeter according to claim 1, wherein
the semiconductor light-receiving element is mounted on a surface of the film on the ring-shaped base material side, and
the film has, at least in a region where the semiconductor light-receiving element is mounted, a property of transmitting the light emitted by the semiconductor light-emitting element, and the semiconductor light-receiving element receives the light which is emitted from the semiconductor light-emitting element and passes through the finger and the film.

3. The probe for a pulse oximeter according to claim 2, wherein
a surrounding of the sealing material that seals the surrounding of the semiconductor light-receiving element is covered with a light-reflective resin.

4. The probe for a pulse oximeter according to claim 3, wherein
an interface between the sealing material and the light-reflective resin is partially inclined.

5. The probe for a pulse oximeter according to claim 1, wherein
the semiconductor light-receiving element is mounted on a surface of the film on the finger side.

6. The probe for a pulse oximeter according to claim 1, wherein
a region of the film on which the semiconductor light-emitting element is mounted or a region on which the semiconductor light-receiving element is mounted is folded to be double layered.

7. The probe for a pulse oximeter according to claim 1, wherein
the electrodes of the semiconductor light-emitting element and the semiconductor light-receiving element are bonded to the wiring on the film by a bonding material, and
the bonding material is a sintered body of metal particles.

8. The probe for a pulse oximeter according to claim 1, wherein
the ring-shaped base material is light-shielding.

9. The probe for a pulse oximeter according to claim 1, wherein
a metal film is disposed on a surface of the ring-shaped base material.
